(19) United States
(12) Patent Application Publication
(10) Pub. No.: US 2026/0248760 A1
(43) Pub. Date: Aug. 27, 2026

(54) METHODS FOR TREATING OPHTHALMIC DISEASES OR DISORDERS

(71) Applicant: Kiora Pharmaceuticals GmbH, Vienna (AT)

(72) Inventors: Romana Seda-Zehetner, Vienna (AT); Eric Daniels, Camberwell, VIC (AU); Brian M. Strem, Encinitas, CA (US)

(21) Appl. No.: 19/546,900

(22) Filed: Feb. 23, 2026

Related U.S. Application Data
(60) Provisional application No. 63/762,448, filed on Feb. 24, 2025.

(51) Int. Cl. A61K 31/381; A61K 9/00; A61K 9/08; A61K 47/02; A61K 47/26; A61P 27/02

(52) U.S. Cl. CPC A61K 31/381; A61K 9/0048; A61K 9/08; A61K 47/02; A61K 47/26; A61P 27/02

(57) ABSTRACT

The present disclosure relates to the use of, and to methods of using, DHODH inhibitor compounds, pharmaceutically acceptable salts thereof, and pharmaceutical compositions including a DHODH inhibitor compound for the treatment of ocular diseases. The ocular diseases include, but are not limited to, proliferative vitreoretinopathy, cystoid macular edema, central serous retinopathy, diabetic retinopathy, endophthalmitis, epiretinal membrane, and toxoplasmosis. The present disclosure also relates to the use of, and to methods of using, DHODH inhibitor compounds, pharmaceutically acceptable salts thereof, and pharmaceutical compositions including a DHODH inhibitor compound to inhibit glial scar formation or reduce glial scar length.

### INCORPORATION BY REFERENCE TO ANY PRIORITY APPLICATIONS

Any and all applications for which a foreign or domestic priority claim is identified in the Application Data Sheet as filed with the present application are hereby incorporated by reference, in their entirety, under 37 CFR 1.57.

### FIELD

The present application relates to the field of ocular therapeutics and the development thereof for use in humans or animals. Disclosed herein are uses or methods of using a DHODH inhibitor compound for the treatment of ocular diseases including, but not limited to, proliferative vitreoretinopathy and related diseases and conditions.

### BACKGROUND

Proliferative vitreoretinopathy (PVR) is an ocular condition that can lead to vision loss or blindness. PVR occurs after retinal detachment repair surgery, or as a result of trauma to the eyeball (open globe injury). Upon development of PVR, retinal cells multiply and form scar tissue and as the scar tightens the retina is pulled away. Despite advancements in surgical techniques, the incidence of PVR as a complication of retinal detachment remains the most common cause of final failure after retinal detachment repair and a challenge for vitreoretinal surgeons. There is an ongoing need for therapies and improvements for treatment of PVR and related diseases.

### SUMMARY

Some embodiments disclosed herein relate to a method of treating an ocular disorder that can include administering to a subject identified as suffering from the ocular disorder an effective amount of a compound having the structure:

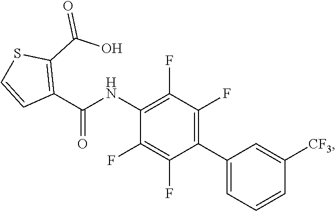 [SMILES: O=C(Nc1c(F)c(F)c(-c2cccc(C(F)(F)F)c2)c(F)c1F)c1ccsc1C(=O)O]

designated KIO-100, or a pharmaceutically acceptable salt thereof, or a pharmaceutical composition that includes an effective amount of the compound, or a pharmaceutically acceptable salt thereof, as described herein. The ocular disorders include, but are not limited to, proliferative vitreoretinopathy, cystoid macular edema, central serous retinopathy, diabetic retinopathy, endophthalmitis, epiretinal membrane, and toxoplasmosis.

Some embodiments disclosed herein relate to a method of inhibiting or reducing glial scar formation, which may include migration, or gliosis that can include administering to a subject identified as at risk for or suffering from glial scar formation or gliosis an effective amount of a compound having the structure:

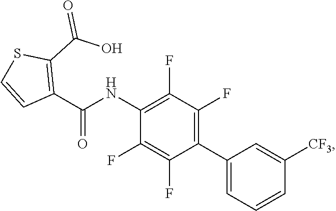 [SMILES: O=C(Nc1c(F)c(F)c(-c2cccc(C(F)(F)F)c2)c(F)c1F)c1ccsc1C(=O)O]

or a pharmaceutically acceptable salt thereof, or a pharmaceutical composition that includes an effective amount of the compound, or a pharmaceutically acceptable salt thereof, as described herein.

Disclosed herein are methods of treating an ocular disorder in a subject, the method comprising a step of administering a therapeutically effective amount of a compound having the structure:

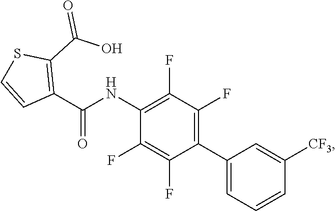 [SMILES: O=C(Nc1c(F)c(F)c(-c2cccc(C(F)(F)F)c2)c(F)c1F)c1ccsc1C(=O)O]

or a pharmaceutically acceptable salt thereof, to the subject in need thereof. The ocular disorder is preferably selected from the group consisting of proliferative vitreoretinopathy, cystoid macular edema, central serous retinopathy, diabetic retinopathy, endophthalmitis, epiretinal membrane, and toxoplasmosis. Most preferably, the ocular disorder is proliferative vitreoretinopathy. In some embodiments, the compound is administered to the subject as a pharmaceutical composition, and the composition comprises the compound and one or more pharmaceutically acceptable excipients. In some embodiments, the pharmaceutically acceptable excipient is selected from the group consisting of surfactants, preservatives, viscosity regulators, pH-adjusting agents, stabilizers, and tonicity regulators.

In some embodiments of these methods, as disclosed herein, the compound comprises from about 0.005% to about 20% by weight of the pharmaceutical composition. In some embodiments, the composition is a liquid. In some embodiments, the concentration of the compound in the composition is from about 0.005 mg/mL to about 1 mg/mL. In some embodiments, the concentration of the compound in the composition is from about 0.01 mg/mL to about 0.5 mg/mL. In some embodiments, the concentration of the compound in the composition is from about 0.02 mg/mL to about 0.2 mg/mL. In some embodiments, wherein the concentration of the compound in the composition is about 0.02 mg/mL. In some embodiments, the concentration of the compound in the composition is about 0.2 mg/mL. In some embodiment, the compound is administered at a dosage of from about 0.1 μg to about 100 μg. In some embodiments, the compound is administered at a dosage of from about 1 μg to 20 μg. In some embodiments, the compound is administered at a dosage of about 1 μg. In other embodiments, the compound is administered at a dosage of about 10 μg. In preferred embodiments, the compound is administered topically or by intravitreal injection.

Also disclosed herein are methods of reducing or inhibiting glial scar formation, which may include migration, or reducing glial scar length in an eye of a subject. These methods comprise administering a therapeutically effective amount of a compound having the structure:

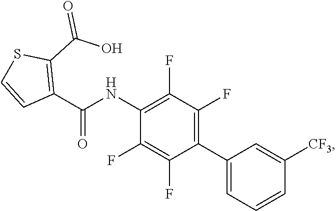 [SMILES: O=C(Nc1c(F)c(F)c(-c2cccc(C(F)(F)F)c2)c(F)c1F)c1ccsc1C(=O)O]

designated KIO-100, or a pharmaceutically acceptable salt thereof, to the subject in need thereof. In some embodiments, the compound is administered to the subject as a pharmaceutical composition comprising the compound and one or more pharmaceutically acceptable excipients. In some embodiments, the compound is administered to the subject as a pharmaceutical composition, and the composition comprises the compound and one or more pharmaceutically acceptable excipients. In some embodiments, the pharmaceutically acceptable excipient is selected from the group consisting of surfactants, preservatives, viscosity regulators, pH-adjusting agents, stabilizers, and tonicity regulators.

In some embodiments of these methods, as disclosed herein, the compound comprises from about 0.005% to about 20% by weight of the pharmaceutical composition. In some embodiments, the composition is a liquid.

In some embodiments, the concentration of the compound in the composition is from about 0.005 mg/mL to about 1 mg/mL. In some embodiments, the concentration of the compound in the composition is from about 0.01 mg/mL to about 0.5 mg/mL. In some embodiments, the concentration of the compound in the composition is from about 0.02 mg/mL to about 0.2 mg/mL. In some embodiments, the concentration of the compound in the composition is about 0.02 mg/mL. In some embodiments, the concentration of the compound in the composition is about 0.2 mg/mL. In some embodiments, the compound is administered at a dosage of from about 0.1 μg to about 100 μg. In some embodiments, the compound is administered at a dosage of from about 1 μg to about 20 μg. In some embodiments, the compound is administered at a dosage of about 1 μg. In some embodiments, the compound is administered at a dosage of about 10 μg. In preferred embodiments, the compound is administered topically or by intravitreal injection.

There are other embodiments described in greater detail below.

### BRIEF DESCRIPTION OF THE DRAWINGS

FIG. 1 shows representative widefield fluorescent microscope images used to quantify isolectin B4 positive cells for Group 1 (vehicle), Group 2 (1 μg/eye KIO-100), and Group 3 (10 μg/eye KIO-100).

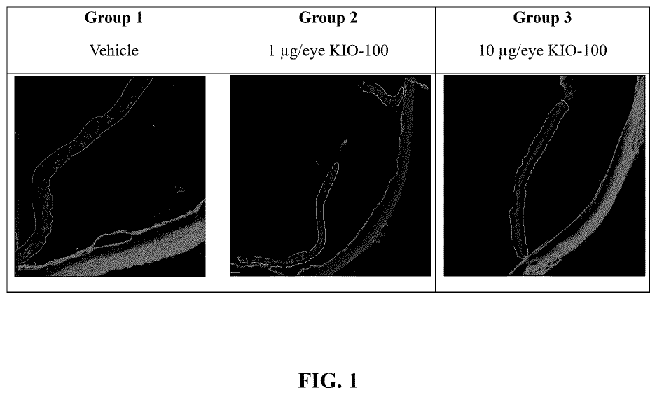

FIG. 2 shows representative 20× widefield fluorescent microscope images used to identify visible scarring and quantify the lengths of induvial scars for Group 1 (vehicle), Group 2 (1 μg/eye KIO-100), and Group 3 (10 μg/eye KIO-100). Brackets indicate length of the scar.

FIG. 3 shows a plot of the individual and mean glial scar lengths for Group 1 (vehicle) and Group 2 (1 μg/eye KIO-100). No scars were measured for Group 3 (10 μg/eye KIO-100) due to a complete absence of glial scarring.

### DETAILED DESCRIPTION

The following description provides context and examples but should not be interpreted to limit the scope of the disclosure covered by the claims that follow in this specification or in any other application that claims priority to this specification. No single component or collection of components is essential or indispensable. For example, in some embodiments one or more variables may be omitted. Any feature, structure, component, material, step, or method that is described and/or illustrated in any embodiment in this specification can be used with or instead of any feature, structure, component, material, step, or method that is described and/or illustrated in any other embodiment in this specification.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. To the extent publications and patents or patent applications incorporated by reference contradict the disclosure contained in the specification, the specification is intended to supersede and/or take precedence over any such contradictory material.

In the event that a plurality of definitions are provided herein for a given term, those in this section prevail unless stated otherwise.

A “formulation” or the like means a composition that one can administer to a subject, e.g., human or animal. Formulations are suitable for human or veterinary applications and would typically have expected characteristics for the formulation, e.g., parenteral formulations for human use would usually be sterile solutions or suspensions.

An “excipient”, “carrier”, “diluent”, “pharmaceutically acceptable carrier” or similar terms mean one or more component(s) or ingredient(s) that is acceptable in the sense of being compatible with the other ingredients in the disclosed compositions or formulations and not overly deleterious to the patient, animal, tissues or cells to which the formulation is to be administered.

As used herein, a “carrier” refers to a compound, particle, solid, semi-solid, liquid, or diluent that facilitates the passage, delivery and/or incorporation of a compound to cells, tissues and/or bodily organs.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline.

The term “excipient” has its ordinary meaning as understood in light of the specification, and refers to inert substances, compounds, or materials added to a pharmaceutical composition to provide, without limitation, bulk, consistency, stability, binding ability, lubrication, disintegrating ability etc., to the composition. Excipients with desirable properties include but are not limited to preservatives, co-solvents, adjuvants, stabilizers, solvents, buffers, diluents, solubilizing agents, detergents, surfactants, chelating agents, antioxidants, viscosity regulators, pH-adjusting agents, penetration enhancers, tonicity regulators, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, hydroxymethyl cellulose, hydroxyethyl cellulose, hydroxypropyl methylcellulose, methylcellulose, carboxymethylcellulose, polyvinylpyrrolidone, hyaluronic acid or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

As used herein, “subject,” “host,” “patient,” and “individual” are used interchangeably and shall be given their ordinary meaning in the art and shall also refer to an organism that has cancer and/or leukemia. This includes mammals, e.g., a human, a non-human primate, ungulates, canines, felines, equines, mice, rats, and the like. The term “mammal” includes both human and non-human mammals.

As used herein, “DHODH inhibitor” refers to an agent that detectably reduces the activity of dihydroorotate dehydrogenase (“DHODH”). Measurement of DHODH activity may be performed by, but is not limited to, detecting conversion of dihydroorotate (DHO) to orotate, detecting dihydroorotate detecting depletion of uridine.

The term “therapeutically effective amount” refers to the amount of active pharmaceutical ingredient necessary to provide a therapeutically relevant pharmacologic result. In practice, the therapeutically effective amount will vary widely depending on the severity of the disease condition, the age of the subject, and undesired side effects. As used herein, the term “effective amount” is broader than the term “therapeutically effective amount,” and refers to an amount of active pharmaceutical ingredient necessary to provide any pharmacologic result.

The terms “treatment,” “treating,” “treat,” and the like shall be given their ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. The terms “treatment,” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt (for example, ammonium or triethylammonium salt), an alkali metal salt, such as a lithium, a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C1-C7 alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof. In addition, the term “comprising” is to be interpreted synonymously with the phrases “having at least” or “including at least”. When used in the context of a compound or composition, the term “comprising” means that the compound or composition includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality.

Where a range of values is provided, it is understood that the upper and lower limit, and each intervening value between the upper and lower limit of the range is encompassed within the embodiments.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

### Methods of Use

Some embodiments disclosed herein relate to selecting a subject or patient in need. In some embodiments, a patient is selected who is in need of treatment, inhibition, amelioration, prevention or slowing of proliferative vitreoretinopathy or associated diseases or conditions, including but not limited to, cystoid macular edema, central serous retinopathy, diabetic retinopathy, endophthalmitis, epiretinal membrane, and toxoplasmosis. In some embodiments, a patient is selected who is in need of treatment, inhibition, reduction, amelioration, prevention or slowing of gliosis or glial scar formation, which may include migration. In some embodiments, a subject can be selected who has previously been treated for the disease or condition described herein. In some embodiments, a subject can be selected who has previously been treated for being at risk for the disease or condition described herein. In some embodiments, a subject can be selected who has developed a recurrence of the disease or condition described herein. In some embodiments, a subject can be selected who has developed resistance to therapies for the disease or condition described herein. In some embodiments, a subject can be selected who may have any combination of the aforementioned selection criteria.

The terms “treating,” “treatment,” “therapeutic,” or “therapy,” as used herein, have their ordinary meanings as understood in light of the specification, and do not necessarily mean total cure or abolition of the disease or condition. The term “treating” or “treatment” as used herein (and as well understood in the art) also means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

Some embodiments described herein relate to a method of treating, inhibiting, ameliorating, preventing, or slowing the disease or condition described herein. In some embodiments, the methods include administering to a subject identified as suffering from the disease or condition described herein an effective amount of a compound, or a pharmaceutically acceptable salt thereof, described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating, inhibiting ameliorating, preventing, or slowing the disease or disorder described herein. Still other embodiments described herein relate to the use of a compound, or a pharmaceutically acceptable salt thereof, as described herein or a pharmaceutical composition that includes an effective amount of a compound, or a pharmaceutically acceptable salt thereof, as described herein for treating, inhibiting ameliorating, preventing, or slowing the disease or condition described herein.

Some embodiments described herein relate to a method for inhibiting or reducing gliosis or glial scar formation that can include administering to a subject as suffering from gliosis, or at risk of gliosis or glial scar formation with an effective amount of a compound, or a pharmaceutically acceptable salt thereof, described herein, or a pharmaceutical composition that includes an effective amount of a compound, or a pharmaceutically acceptable salt thereof, described herein. Other embodiments described herein relate to the use of an effective amount of a compound, or a pharmaceutically acceptable salt thereof, described herein, or a pharmaceutical composition that includes an effective amount of a compound, or a pharmaceutically acceptable salt thereof, described herein in the manufacture of a medicament for inhibiting or reducing gliosis or glial scar formation. Still other embodiments described herein relate to an effective amount of a compound, or a pharmaceutically acceptable salt thereof, described herein, or a pharmaceutical composition that includes an effective amount of a compound, or a pharmaceutically acceptable salt thereof, described herein for inhibiting or reducing gliosis or glial scar formation.

Those of skill in the treatment of such diseases or conditions could determine the effective therapeutic amount from test results. In some embodiments, the compound can be administered at a dosage from about 0.1 μg to about 100 μg, and in particular about 1 μg to about 20 μg, more preferably about 1 μg to about 10 μg, or any dosage or dosage range spanning the aforementioned values. In certain embodiments, the compound is administered at a dosage of about 1 μg. In another embodiment, the compound is administered at a dosage of about 10 μg.

In some embodiments, methods can include local administration to the eyes of the subjects to be treated to mitigate potential negative side effects of administration via systemic circulation. Accordingly, the methods described herein may include administration of pharmaceutical compositions formulated for a local ocular route such as periocular, conjunctival, subtenon, intracameral, intravitreal, intraocular, subretinal, subconjunctival, retrobulbar, suprachoroidal or intracanalicular injections; by direct application to the eye using a catheter or other placement devices, such as a retinal pellet, intraocular insert, suppository, intra- or periocular implants (intrascleral, periscleral, episcleral), intravitreous implants or suprachoroidal implants or particles or polymeric composition, or any releasing systems such as emulsions, solid non-biodegradable or degradable implants or tablets, mini pumps, or topical formulations or by a slow release device in the cul-de-sac or implanted adjacent to the sclera (transscleral) or in the sclera (intrascleral) or suprachoroidal or within the eye. Intracameral injection may be through the cornea into the anterior chamber to allow the agent to reach the trabecular meshwork. Intracanalicular injection may be into the venous collector channels draining Schlemm's canal or into Schlemm's canal.

In some embodiments, the method may include administration of the compound, pharmaceutically acceptable salt thereof, or pharmaceutical composition that includes an effective amount of the compound, into the eye, e.g. as intravitreal injection or implant. In other embodiments, the compound, pharmaceutically acceptable salt thereof, or pharmaceutical composition that includes an effective amount of the compound, described herein may be applied topically (e.g., in the form of eye drops) between once daily and up to eight times a day. Some patients may benefit from regular application of the formulation, such as for at least about 3 days, at least about 10 days, at least about 1 month, at least about 3 months or any range spanning the aforementioned values. In some embodiments, administration via injection may done less frequently compared to topical administration.

Delivery of drugs to the posterior segment of the eye is difficult and it is very difficult to maintain a drug concentration in those tissues. In some embodiments, the compound can be administered via intravitreal injection. In some embodiments of the methods described herein, intravitreous injection can deliver a larger amount of the drug to be delivered to the posterior segment of the eye compared to the amount that could be delivered by intravenous injection or oral administration. In some embodiments, intravitreous injection of a compound avoids or mitigates side effects caused by systemic circulation of a compound administered via intravenous injection or oral administration.

In some embodiments of the methods described herein, intravitreal injections release the DHODH inhibitor continuously over a longer period of time at a release rate which ensures continuous therapeutic concentration of the drug at the desired site of action of. Examples for such formulations are PLGA nano-spheres or PLGA microspheres. In such formulations, the DHODH inhibitor is embedded in small spheres of PLGA polymers and slowly released within the eye while the PLGA is degraded. Depending on the ratio of lactic acid and glycolic acid and the degree of crosslinking of the polymer and depending on the manufacturing process of the spheres, the complete drug can be released within 1 week to 1 month, within 6 months, or within 12 months or longer periods. A slow release of therapeutically active amounts of the DHODH inhibitor from the formulation after intravitreal injection can reduce the intervals between single injections into the eye. Similar results can be obtained with implants.

As used herein, the term “combination therapy” is intended to define therapies which comprise the use of a combination of two or more pharmaceutical compounds/agents or therapies. Thus, references to “combination therapy”, “combinations” and the use of compounds/agents “in combination” in this application may refer to compounds/agents that are administered as part of the same overall treatment regimen. As such, the dosage or timing of each of the two or more compounds/agents may differ: each may be administered at the same time or at different times. Accordingly, the compounds/agents of the combination may be administered sequentially (e.g. before or after) or simultaneously, either in the same pharmaceutical formulation (i.e. together), or in different pharmaceutical formulations (i.e. separately). Each of the two or more compounds/agents in a combination therapy may also differ with respect to the route of administration.

### Compositions and Formulations for Administration

In some embodiments of the methods described herein, the active ingredient (e.g. DHODH inhibitor compound) may be combined with one or more pharmaceutically acceptable excipients, and/or optionally sustained release matrices, such as biodegradable polymers, to form a pharmaceutical composition for administration.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

In some embodiments, the pharmaceutical composition contains vehicles which are pharmaceutically acceptable for a formulation capable of being injected into the eye. These may be, in particular, isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical compositions suitable for injectable use in the eye include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and may be preserved against the contaminating action of microorganisms, such as bacteria, virus and fungi.

Solutions comprising a DHODH inhibitor compound as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations may contain a preservative to prevent the growth of microorganisms.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, benzalkonium chloride, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions for the eyes are prepared by incorporating the active ingredients of the invention in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as it is therapeutically effective. The formulations are easily administered in a variety of dosage forms, such as the type of injectable solutions described above, but drug release capsules and the like may also be employed.

For ophthalmic delivery, the active ingredient may be combined with one or more pharmaceutically acceptable excipients, including, but not limited to, ophthalmologically acceptable preservatives, co-solvents, surfactants, viscosity regulators, pH-adjusting agents, stabilizers, penetration enhancers, tonicity regulators, buffers, sodium chloride, or water as a pharmaceutical composition. In some embodiments, the pharmaceutical composition can be a liquid. In some embodiments, the composition can be an aqueous, sterile ophthalmic suspension or solution. Solution formulations may be prepared by dissolving the active ingredient in a physiologically acceptable isotonic aqueous buffer. Further, the solution may include an acceptable surfactant to assist in dissolving the active ingredient. Viscosity building agents, such as hydroxymethyl cellulose, hydroxyethyl cellulose, hydroxypropyl methylcellulose, methylcellulose, carboxymethylcellulose, polyvinylpyrrolidone, hyaluronic acid, or the like may be added to the compositions of the present invention to improve the retention of the compound.

In some embodiments, the compound can be from about 0.005% to about 20% by weight of the composition. In other embodiments, the compound can be from about 0.01 to about 10% by weight of the composition. In still other embodiments, the compound can be from about 0.1 to about 1% by weight of the composition or any value spanning the aforementioned ranges.

In some embodiments, the concentration of the compound in the composition, ophthalmic suspension or solution is from about 0.005 mg/mL to about 1 mg/mL, preferably 0.01 mg/mL to about 0.05 mg/mL, and more preferably is from about 0.02 mg/mL to about 0.2 mg/mL, or any range spanning the aforementioned values. In some embodiments, the concentration of the compound in the composition, suspension, or solution is about 0.02 mg/mL. In other embodiments, the concentration of the compound in the composition, suspension, or solution is about 0.2 mg/mL.

In order to prepare a sterile ophthalmic ointment formulation for administration according to the methods described herein, the active ingredient can be combined with a preservative in an appropriate vehicle, such as mineral oil, liquid lanolin, or white petrolatum. Sterile ophthalmic gel formulations may be prepared by suspending the active ingredient in a hydrophilic base prepared from the combination of, for example, CARBOPOL®-940 (BF Goodrich, Charlotte, NC), or the like, according to methods known in the art. VISCOAT® (Alcon Laboratories, Inc., Fort Worth, TX) may be used for intraocular injection, for example. In other embodiments, compositions may contain penetration enhancing agents such as Cremophor and TWEEN® 80 (polyoxyethylene sorbitan monolaurate, Sigma Aldrich, St. Louis, MO), in the event the active ingredient is less penetrating in the eye.

In some embodiments, the pharmaceutical composition for administration according to the methods described herein may be an ophthalmic drop formulation. The eye drop is provided in any formulation generally used, for example, in the form of an aqueous eye drop such as aqueous eye drop solution, aqueous eye drop suspension, viscous eye drop solution, solubilized eye drop solution and the like, or in the form of a non-aqueous eye drop such as a nonaqueous eye drop solution, non-aqueous eye drop suspension and the like. When the composition for administration is prepared as an aqueous eye drop, it preferably contains an additive, which is usually used in an aqueous eye drop. The examples of such an additive include preservatives, isotonic agents, buffering agents, stabilizer, pH regulators or the like.

In other embodiments, the active ingredients for administration according to the methods described herein are delivered through a biodegradable ocular implant. The implants can be formed in a manner that the active ingredient is homogenously distributed or dispersed throughout the biodegradable polymer matrix. Additionally, the implants can be formed to release the active ingredient into an ocular region of the eye over various time periods. Thus, the active ingredient can be released from implants made according to the present invention for a period of time of, for example, 30-200 days.

In some embodiments, the active ingredient for administration in the methods described herein, can comprise from about 10% to about 90% by weight of the implant. In some embodiments, the agent is from about 40% to about 80% by weight of the implant. In another embodiment, the agent comprises about 60% by weight of the implant.

In some embodiments, the active ingredient can be homogeneously dispersed in the biodegradable polymer of the implant. The implant can be made, for example, by a sequential or double extrusion method. The selection of the biodegradable polymer used can vary with the desired release kinetics, subject tolerance, the nature of the disease to be treated, and the like. Polymer characteristics that are considered include, but are not limited to, the biocompatibility and biodegradability at the site of implantation, compatibility with the active ingredient of interest, and processing temperatures. The biodegradable polymer matrix usually comprises at least about 10, at least about 20, at least about 30, at least about 40, at least about 50, at least about 60, at least about 70, at least about 80, or at least about 90 weight percent of the implant. In some embodiments, the biodegradable polymer matrix comprises about 40% to 50% by weight of the implant.

Biodegradable polymers which can be used include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be crosslinked or non-crosslinked. If crosslinked, they are usually not more than lightly crosslinked, and are less than 5% crosslinked, usually less than 1% crosslinked. Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic) acid (PLGA) copolymer may be 0-100%, about 15-85%, about 25-75%, or about 35-65%. In some embodiments, 25/75 PLGA and/or 50/50 PLGA copolymers are used. In other embodiments, PLGA copolymers are used in conjunction with polylactide polymers or polyurethanes.

Other agents may be employed in the formulation for administration for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The formulation for administration according to the methods described herein may further comprise an antiphlogistic or antibiotic agent like azithromycin, specifically an agent with additional anti-inflammatory properties, specifically histamine antagonists or non-steroidal anti-inflammatory drugs.

The formulation for administration according to the methods described herein may also be formulated as depot formulation, which provides continuous or prolonged administration.

According to a further embodiment, the formulation can be used in preventing recurrence of an ocular disease or condition in a subject.

According to a further embodiment, the formulation can be used in preventing recurrence of ocular inflammations in a subject.

Preferably, the formulations for administration according to the methods described herein are stable in a wide range of temperatures.

In some embodiments, the formulation can be packaged in a hermetically sealed container such as an ampoule or sachette indicating the quantity of formulation.

In some embodiments, the formulation is supplied as a liquid. In another embodiment, as a dry sterilized lyophilized powder or water free concentrate, or as dry and sterile nano- or microspheres of drug-containing PLGA copolymers in a hermetically sealed container and can be reconstituted, e.g., with water or saline to the appropriate concentration for administration to a subject.

In an alternative embodiment, the composition for administration according to the methods described herein is supplied in liquid form in a hermetically sealed container indicating the quantity and concentration of the composition.

### EXAMPLES

Additional embodiments are disclosed in further detail in the following representative examples, which are not in any way intended to limit the scope of the claims. The following examples are provided for the guidance of the reader, and do not include detailed descriptions of conventional methods that are well known to those of ordinary skill in the art.

### Example 1: Efficacy of KIO-100 in a Rabbit Model of Retinal Detachment

The procedures described herein abided by the Institutional Animal Care and Use Committee SOPs.

Eighteen male Dutch Belted rabbits were obtained from Envigo (Denver, PA). Animals were 4-6 months old and weighted 1.5-3.5 kg on Day 1. Upon arrival, animals were quarantined and acclimated to the study environment for 1 week and physically examined to ensure they were in good health and suitable for study participation. Animals received Purina® Hi Fiber Lab Rabbit Diet #5P25 and water ad libitum. Ocular examinations were conducted by a veterinary ophthalmologist using a slit lamp biomicroscope and indirect ophthalmoscope and lens to evaluate anterior and posterior segments and confirm normal ocular baselines.

Prior to dosing, six animals were randomly assigned to each of three study groups (Group 1: Vehicle control; Group 2:1 μg/eye KIO-100; Group 3:10 μg/eye KIO-100) according to standard operating procedures. Animals were uniquely identified by corresponding cage card number and ear tag. A vehicle control stock solution was composed of 9.99% sucrose (w/w), 0.02% 1M NaOH (w/w) and 1.0% 0.1M phosphate buffer (w/w) in water for injection (WFI) and was prepared by mixing of 44.476 g of an aqueous sucrose/NaOH solution with 0.5 g of 0.1 M phosphate buffer. The solution was stirred for 15 min and the pH was adjusted with 0.1 M HCl or 0.1 M NaOH to 7.2±0.2. Water for injection (WFI) was added to a final weight of 50 g and the osmolarity was measured to be between 270-330 mOsmol/kg. A 0.5 mg/mL stock solution of KIO-100, composed of 0.05% KIO-100 (w/w), 9.99% sucrose (w/w), 0.02% 1M NaOH and 1.0% of 0.1M phosphate buffer (w/w) in WFI was prepared by dissolving 0.024 g KIO-100 in 44.476 g of an aqueous sucrose/NaOH solution by continuous stirring over 12 to 24 hours at ambient temperature. After complete dissolution of KIO-100, 0.5 g of 0.1M phosphate buffer was added and pH was adjusted to 7.2±0.2 with 0.1 M HCl or 0.1 M NaOH. WFI was added to a final weight of 50 g and the osmolarity was measured to be between 270-330 mOsmol/kg. The 0.5 mg/ml KIO-100 stock solution was diluted with 0.9% sterile saline under sterile conditions to prepare dose concentrations of 0.02 mg/mL (low/Group 2) and 0.20 mg/mL (high/Group 3). The Vehicle control stock solution was also diluted under sterile conditions using 0.9% sterile saline (in accordance with high/Group 3 dilution ratio) and used for dosing of Group 1. The final formulations were sterile filtered 0.2 μg filter with PES membrane. All diluted solutions were used within 24 hours of preparation.

Prior to surgery, the animals received buprenorphine extended-release (0.12 mg/kg, SQ); animals designated as spares received buprenorphine sustained-release SQ on Day 1 if needed as replacements. Eyes were dilated with 1% tropicamide HCl and animals were sedated with a ketamine/dexmedetomine cocktail (10/0.1 mg/kg IM) administered intramuscularly (IM). Following induction of anesthesia, eyelashes were trimmed, and ~5% betadine solution on autoclaved sterile gauze was used to clean the periocular area of each eye and to irrigate the ocular surface and conjunctival cul-de-sacs (1 minute application time), followed by rinsing with sterile eyewash. Then, 10% phenylephrine HCl and 0.5% proparacaine HCl were applied to the corneal surface at least 2 minutes apart. Each animal was positioned beneath the surgical microscope with the OS upwards. A surgical drape and eyelid speculum were placed.

The induction agent, 0.25% hyaluronic acid (HA) was prepared from 1.8% sodium hyaluronate diluted in sterile balanced salt solution to 0.25%. The 0.25% HA was loaded into a a MicroDose™ SF Injection Kit (MedOne, #3315) and connected to an extendible PolyTip® SR injection cannula (25G×28 mm cannula with 38G [5 mm length] tip; MedOne, #3247). The MedOne polymeric tip was extended, and the needle was primed with approximately 240 μL (PolyTip) of (HA)). The polymeric tip was then retracted, and the needle and syringe pair were moved to the surgical field. The needle and syringe pair remained horizontal and in the same plane prior to injection. The MicroDose Injector was attached to a Viscous Fluid Control Pak (Alcon, Cat #8065750957) to allow foot pedal-actuated injections at a controlled rate via the Constellation system. Care was taken to avoid excessively kinking or twisting the extension tubing. If solution was observed at the needle tip, it was carefully wiped with sterile gauze. Before attempting to inject animals, a single dose was piloted to ensure adequate pressure buildup throughout the system, ensuring reliable dose delivery.

Two (2) 25G non-valved ports were placed at the pars plana to facilitate needle and light source entry into the vitreous. Wet field cautery was used to prepare the sites intended for the insertion of 25G ports. A small incision in the clear cornea was made using a 1.2 mm keratome to release pressure in the eye as needed. The RESIGHT 700 fundus viewing system (Zeiss) or a lens placed on the cornea was used to visualize the posterior segment. A local retinal detachment was created in the retina and 0.25% HA was administered inferior to the optic disc/medullary ray via SR injection using the primed MedOne injector. Approximately 180-260 μL of the prepared HA was injected at a gradual rate. After injection, the needle was held in place for at least 5 seconds and the retinotomy site was extended (2-4 mm) to allow for simulation of a rhegmatogenous retinal detachment for fluid exchange in the subretinal injection. While the animals were sedated and with the ports in place, intravitreal injection of Vehicle or KIO-100 (50 μL/eye-1 μg/eye for Group 2 and 10 μg/eye for Group 3) was administered using a 25-gauge, 1-inch needle attached to a BD Luer-Lok syringe. The IVT dose was administered close to the site of the retinotomy. The conjunctiva was closed with suture. Neomycin and polymyxin b sulfates and gramicidin topical solution were topically applied to the eye. Following model induction, color fundus imaging using RetCam3 (Natus) of the posterior section of the eye was used to visualize and assess the success of the retinal detachment. If needed, a wire eyelid speculum was placed, and 1% tropicamide HCl, 0.5% proparacaine HCl, and eye lubricant were applied.

Cage-side clinical observations were performed twice daily, with particular attention paid to the eyes. All animals were observed twice daily for mortality, abnormalities, and signs of pain or distress. At all observation timepoints, all animals were bright, alert, and responsive with no significant findings, with the following exceptions:

At baseline and on Day 3, ocular examinations (OEs) were performed using a slit lamp biomicroscope and indirect ophthalmoscope to evaluate anterior and posterior segments clinical observations for all eyes. A topical mydriatic (1% tropicamide HCl) was administered following the anterior segment evaluation to facilitate examination of the posterior segment. The semiquantitative preclinical ocular toxicology scoring (SPOTS) ocular grading system as described in Eaton et al., J. Ocul. Pharmacol. Ther. 2017 December; 33 (10): 718-734, which is incorporated herein by reference in its entirety, was used for scoring. All animals with normal baseline OEs were considered eligible for enrollment into the study. Animals were not tranquilized for examinations.

All baseline OEs were unremarkable. On Day 3, the OEs of all right eyes (ODs) were normal.

On Day 3, total OE scores in left eyes (OSs) for Group 1 were 4-6, with all eyes having mild to moderate conjunctival hyperemia (scores of 1-2 out of 3), mild to moderate conjunctival chemosis (scores of 1-3 out of 4), and mild to moderate aqueous flare (1-2 out of 3); 3 eyes had mild vitreous haze (scores of 1 out of 4); and all eyes had a large inferior retinal detachment, with an anterior vitreal hemorrhage also visible in one OS.

In Group 2 OSs, total OE scores were 2-8; all eyes had mild to moderate conjunctival hyperemia and mild conjunctival chemosis and 5 out of 6 eyes had mild aqueous flare. Of the eyes with aqueous flare, 1 of those eyes also had mild iris values (score of 1 out of 4) and mild vitreous haze; 1 had mild cellular flare (score of 1 out of 4), moderate vitreous haze (score of 2 out of 4), and mild vitreous cells (score of 1 out of 4); 1 eye had mild vitreous haze, and 1 eye had mild vitreous haze and mild vitreous cells. All Group 2 OSs had a large inferior retinal detachment.

In Group 3 OSs, total OE scores were 4-7. All eyes had mild to moderate conjunctival hyperemia, mild conjunctival chemosis, and mild to moderate aqueous flare; 1 eye also had mild vitreous haze and mild vitreous cells, 1 eye also had mild iris values and mild vitreous cells, and 1 eye also had mild vitreous cells. All Group 3 OSs had an inferior retinal detachment: 4 were large and 1 was medium.

On Day 8, animals were sedated with a ketamine/dexmedetomidine cocktail (10-15/0.01-0.2 mg/kg IM) and euthanized with an overdose of sodium pentobarbital administered intravenously or intracardiac, followed by auscultation to ensure death. Following euthanasia, eyes designated for cryosectioning were enucleated and marked with a metallic Sharpie® superiorly at the 12 o'clock position. A 3-mm incision was made at the limbus and the eye was placed in 4% paraformaldehyde (PFA) in phosphate-buffered saline for 24 h at room temperature. The anterior segment and lens were dissected away, and the vitreous humor removed. Eyes were cryoprotected in a gradient of sucrose (10-30%, 1 hour each) and embedded in OCT such that the optic nerve was facing down/anterior segment opening up; nasal and temporal orientation were tracked. Eyes were frozen on dry ice and stored at −80° C. until sectioning.

To survey the retinal detachment region, the surface closest to the area of detachment was sectioned first (nasal or temporal) and sets of contiguous ribbons of sections (n=3 sections/slide) were captured onto two slides every 80 μm within the detachment area (~50 slides total), using the tissue grossing images to ascertain area of detachment. Every fifth slide (approximately 8-10 slides per eye) was stained. Slides were treated at room temperature with a permeabilization and blocking buffer for 1 h (0.05% Triton X-100 diluted in PBS containing 2% BSA and 2% NDS). Slides were incubated with an antibody dilution buffer (PBS containing 2% BSA and 2% NDS) overnight at 4° C. with 1/1000 chicken anti-vimentin primary antibodies or for 2-3 h at room temperature with 1/200 donkey anti-chicken Cy2, 1/100 Isolectin I-B4 DyLight 649 and 1/1,000 DAPI secondary antibodies.

Slides were imaged on a widefield fluorescent microscope equipped with plan apochromatic objectives. For each enrolled eye, a 4× tiled overview and 20× higher magnification image was captured where immune cells (Isolectin I-B4 positive cells) and scarring were observed and the number and length of scars were measured along with the number of immune cells.

TABLE 1

| Group No. | Cells/mm² (average/eye) |
|---|---|
| 1 | 0.4584 |
| 2 | 0.3591 |
| 3 | 0.4277 |

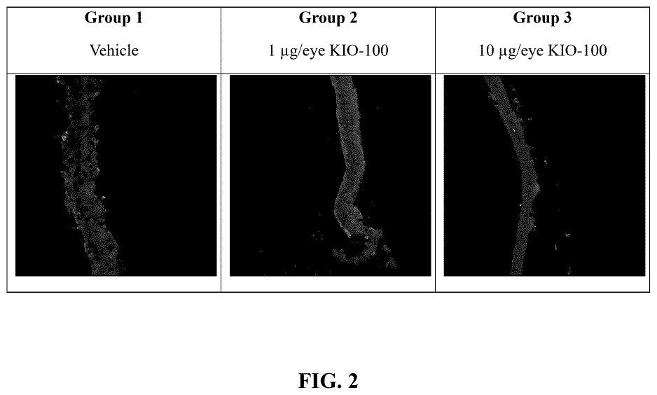

FIG. 2 shows representative image used to quantify the number of immune cells (Isolectin I-B4 positive cells) for each Group as shown in Table 1. The total number of isolectin B4 positive cells were quantified using QuPath. A pixel intensity threshold was applied to all images in the GFP channel to create an annotation around the retina (green outline in FIG. 2). Next, the cell parameters were defined using the Cy5 channel to detect all Isolectin I-B4-positive cells (red outline within the retina annotation). The number of positive cells were automatically counted in QuPath after running the cell detection script. At least three measurements per eye and at least 3 animals per group were measured. The number of cells positively detected in each section was normalized by dividing the total area of the retina annotation to provide a final cells/mm² measurement. As shown in Table 1, the immune cell observations were similar between groups.

TABLE 2

| | | | Total No. |
|---|---|---|---|
| Group No. | Measurable Scars | Non-Measurable Scars | of Scars |
| 1 | 16 | 4 | 20 |
| 2 | 7 | 2 | 9 |
| 3 | 0 | 0 | 0 |

Total scar counts, as shown in Table 2, were determined for each eye, as well as the occurrence of scarring within damaged tissue. The following characteristics must have been met to qualify as a glial scar: 1) positive vimentin expression (GFP) without visible, overlapping autofluorescence in the red channel and 2) must be located SR (subretinal-below the photoreceptor cell layer). The number of glial scars per eye were manually counted in the 20× images.

Group 1 (n=6) exhibited a greater total number of scars, including more than double the number of measurable scars, when compared to Group 2 (n=6), which had been treated with low dose KIO-100 (0.02 mg/mL or 1 μg/eye). Also, Group 1 had more medium- to large-sized scars (eight large scars and one medium-sized scar) when compared to Group 2 (only two medium-sized scars). Group 3 (n=5) with high dose KIO-100 (0.20 mg/ml or 10 μg/eye) showed no scarring and less gliosis when compared to Groups 1 and 2.

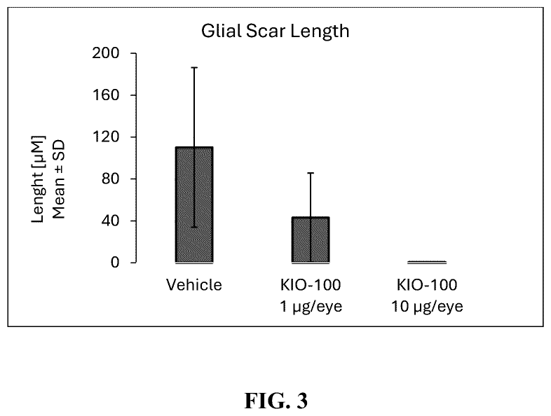

For each enrolled 20× image with visible scarring, the length of individual scars was quantified using ImageJ. FIG. 3 shows representative images for performing glial scar assessment for each of Groups 1, 2, and 3. A segmented line was used to trace the length of the scar and then quantified (in μm). No images were enrolled from Group 3 and scar lengths were not calculated due to a complete absence of glial scarring. Additionally, lesions in areas where the Müller glia cells were degenerative or at the end of a retina break (regions within the bleb where retina was missing) were considered immeasurable. FIG. 4 shows that the average length of individual scars in Group 1 was 110.1 μm, with scar length ranging from 13.9 μm to 242 μm. Scar length was less variable in Group 2 and substantially decreased compared to Group 1, with an average length of 43.2 μm.

Although the foregoing has been described in some detail, by way of illustrations and examples for purposes of clarity and understanding, those of ordinary skill in the relevant art will appreciate and understand that numerous and various modifications can be made without departing from the substance and content of the present disclosure. Therefore, it should be understood that the forms disclosed herein are merely illustrative and are not intended to limit the scope of the present disclosure; the present disclosure covers and includes various modification and alternatives based on the illustrative examples disclosed herein.

1. A method of treating an ocular disorder in a subject, said method comprising administering a therapeutically effective amount of a compound having the structure: 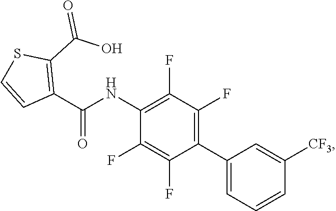 [SMILES: O=C(Nc1c(F)c(F)c(-c2cccc(C(F)(F)F)c2)c(F)c1F)c1ccsc1C(=O)O]
or a pharmaceutically acceptable salt thereof, to the subject in need thereof, wherein the ocular disorder is selected from the group consisting of proliferative vitreoretinopathy, cystoid macular edema, central serous retinopathy, diabetic retinopathy, endophthalmitis, epiretinal membrane, and toxoplasmosis.

2. The method of claim 1, wherein the ocular disorder is proliferative vitreoretinopathy.

3. The method of claim 1, wherein the compound is administered to the subject as a pharmaceutical composition comprising the compound and one or more pharmaceutically acceptable excipients selected from the group consisting of surfactants, preservatives, viscosity regulators, pH-adjusting agents, stabilizers, and tonicity regulators.

4. (canceled)

5. The method of claim 3, wherein the compound comprises from about 0.005% to about 20% by weight of the pharmaceutical composition.

6. The method of claim 3, wherein the composition is a liquid, and wherein the concentration of the compound in the composition is from about 0.005 mg/mL to about 1 mg/mL.

7. (canceled)

8. (canceled)

9. The method of claim 6, wherein the concentration of the compound in the composition is from about 0.02 mg/mL to about 0.2 mg/mL.

10. (canceled)

11. (canceled)

12. The method of claim 1, wherein the compound is administered at a dosage of from about 0.1 μg to about 100 μg.

13. The method of claim 1, wherein the compound is administered at a dosage of from about 1 μg to 20 μg.

14. (canceled)

15. (canceled)

16. The method of claim 1, wherein the compound is administered topically.

17. The method of claim 1, wherein the compound is administered by intravitreal injection.

18. A method of reducing or inhibiting glial scar formation or reducing glial scar length in an eye of a subject, said method comprising administering a therapeutically effective amount of a compound having the structure: 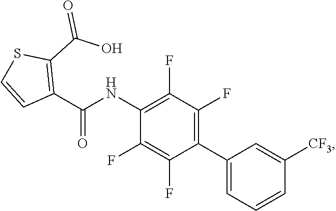 [SMILES: O=C(Nc1c(F)c(F)c(-c2cccc(C(F)(F)F)c2)c(F)c1F)c1ccsc1C(=O)O]
or a pharmaceutically acceptable salt thereof, to the subject in need thereof.

19. The method of claim 18, wherein the compound is administered to the subject as a pharmaceutical composition comprising the compound and one or more pharmaceutically acceptable excipients selected from the group consisting of surfactants, preservatives, viscosity regulators, pH-adjusting agents, stabilizers, and tonicity regulators.

20. (canceled)

21. The method of claim 18, wherein the compound comprises from about 0.005% to about 20% by weight of the pharmaceutical composition.

22. The method of claim 18, wherein the composition is a liquid, and wherein the concentration of the compound in the composition is from about 0.005 mg/mL to about 1 mg/mL.

23. (canceled)

24. (canceled)

25. The method of claim 22, wherein the concentration of the compound in the composition is from about 0.02 mg/mL to about 0.2 mg/mL.

26. (canceled)

27. (canceled)

28. The method of claim 18, wherein the compound is administered at a dosage of from about 0.1 μg to about 100 μg.

29. The method of claim 18, wherein the compound is administered at a dosage of from about 1 μg to about 20 μg.

30. (canceled)

31. (canceled)

32. The method of claim 18, wherein the compound is administered topically.

33. The method of claim 18, wherein the compound is administered by intravitreal injection.

34. The method of claim 18, wherein the glial scar formation includes glial migration.
